(19) United States
(12) Patent Application Publication
(10) Pub. No.: US 2026/0256709 A1
(43) Pub. Date: Sep. 3, 2026

(54) BIODEGRADABLE DENDRITIC PARTICLES FOR SUSTAINED DRUG RELEASE

(71) Applicant: The Regents of the University of Colorado, a body corporate, Denver, CO (US)

(72) Inventors: Charles W. Shields, Longmont, CO (US); Jin Gyun Lee, Broomfield, CO (US)

(21) Appl. No.: 19/165,113

(22) PCT Filed: Mar. 14, 2024
(86) PCT No.: PCT/US2024/020004

§ 371 Date: Sep. 13, 2025

Related U.S. Application Data
(60) Provisional application No. 63/451,987, filed on Mar. 14, 2023.

(51) Int. Cl. A61K 9/51; A61K 45/06

(52) U.S. Cl. CPC A61K 9/5153; A61K 9/5161; A61K 45/06

(57) ABSTRACT

Described herein are methods, systems and compositions for the production and use of a class of highly branched, nanostructured particles, generally referred to herein as dendritic particles (DPs), synthesized from biodegradable materials that are specifically adapted for drug release in vivo.

### STATEMENT OF RELATED CASES

This International PCT application claims the benefit of and priority to U.S. Provisional Application No. 63/451,987, filed Mar. 14, 2023, which is incorporated herein by reference in its entirety.

### STATEMENT OF GOVERNMENT INTEREST

This invention was made with government support under grant number 1R35GM147455 awarded by the National Institutes of Health. The government has certain rights in the invention.

### TECHNICAL FIELD

The present invention relates to the field of material science, and novel compositions and methods for sustained drug delivery. Specifically, the invention is directed to the production soft dendritic particles, and their use in the localized delivery of therapeutic compounds (drugs).

### BACKGROUND

In recent decades, engineered nanoparticles have garnered considerable attention for their high surface area-to-volume ratios, tunable drug release rates, and ability to deliver drugs to target sites deep within the body. However, such traditional nanoparticle drug delivery systems have shown significant technical drawbacks that limit their commercial and therapeutic potential. For example, engineered nanoparticles generally exhibit weak attractive interactions between their surfaces and their cellular targets, such as epithelia, resulting in poor retention times, limiting their benefits in clinical settings. Indeed, numerous studies using nanoparticles for drug delivery have demonstrated that traditional spherical nanoparticles exhibit poor adhesion on targets due to poor interactions between particles and soft tissues. In one study highlighted below, only 0.7 percent of administrated nanoparticles are found in target sites. (See Wilhelm, S., Tavares, A., Dai, Q. et al. Analysis of nanoparticle delivery to tumors. Nat Rev Mater 1, 16014 (2016).

As such, there is a long-felt need for improved engineered nanoparticle systems that can effectively deliver therapeutic compounds locally to target cells and tissues that overcomes the deficiencies in the art. As described below, the present inventors describe novel soft microparticles with nanostructured branches that can be embedded with therapeutic compounds, such as chemotherapeutic agents and other small molecules compounds. The highly dendritic morphology of the particles allows them to strongly adhere to, in particular epithelial tissues, gradually release the embedded therapeutic compounds. Being biodegradable, the engineered nanoparticles of the invention are configured to degrade into biocompatible products under physiologically relevant conditions.

### SUMMARY OF THE INVENTION

In one aspect, the invention includes a novel class of highly branched, nanostructured particles, generally referred to herein as dendritic particles (DPs) also sometime referred to a soft dendritic particles (sDPs), synthesized from biodegradable materials that can be specifically adapted for drug delivery. Due to their extremely high surface areas, the DPs of the invention robustly adhere to biological tissues for sustained periods, whereupon release of therapeutic or prophylactic drugs occurs in a controlled and sustained fashion. As described below, the morphology of the DPs enables robust adhesion to biological surfaces (e.g., epithelial tissues, mucosa, skin, lining of organs) without the use of adhesives. The DPs can slowly release drugs for an extended period without becoming dislodged while breaking down into safe, biocompatible byproducts in physiological conditions.

Additional aspects of the invention include methods and compositions for treating a subject in need thereof. In this preferred embodiment, a therapeutically effective amount of DPs embedded with a therapeutic agent are administered to a subject in need thereof.

Additional aspects of the invention include pharmaceutical compositions for treating a subject in need thereof, the composition including a therapeutically effective amount of DPs embedded with at least one therapeutic agent and a pharmaceutically acceptable carrier, which are administered to a subject in need thereof. Additional aspects of the invention include a kit, including a pharmaceutical composition comprising a therapeutically effective amount of DPs embedded with a therapeutic agent and a pharmaceutically acceptable carrier, a container for holding the composition, and instructions for use. In a preferred aspect, the carrier includes a hydrogel.

Additional aspects of the invention are further described the specification, figures, and claims disclosed herein.

### BRIEF DESCRIPTION OF THE FIGURES

FIG. 1A-B: (A) shows florescence microscopy images of DPs at 200 μm and 100 μm magnification in one embodiment thereof. (B) shows scanning electron microscopy (SEM) images of DPs at 100 μm and 50 μm magnification in one embodiment thereof.

FIG. 2A-B: (A) shows florescence microscopy images of DPs made from 50 kDa PLGA at 100 μm magnification in one embodiment thereof. (B) shows florescence microscopy images of DPs made from 200 kDa PLGA at 100 μm magnification in one embodiment thereof.

FIG. 3A-C: (A) shows the chemical formula for the exemplary therapeutic compound gemcitabine. (B) shows a chart demonstrating the fractional release of gemcitabine from DPs over time in one embodiment thereof. (C) shows a scanning electron microscopy (SEM) image of DPs bound to a mouse bladder epithelium at 100 μm magnification in one embodiment thereof.

FIGS. 4A-C: (A) shows a diagram of the preparation of DPs using fluid flow templating. In highly turbulent flow, precipitation of the PLGA in an antisolvent results in the formation of branched nanofibers around the particle→dendritic particles (DPs) (B-C) image of exemplary DPs at 500 μm and 50 μm magnification using florescence microscopy and SEM, respectively.

FIG. 5A-B: Chitosan functionalization on poly(lactic-co-glycolic acid) (PLGA) surfaces. Chitosan is a polysaccharide and widely used to promote adhesion of biomaterials to cells and soft tissues. (A) as the chitosan concentration increases, zeta potential of PLGA nanoparticles (NPs) switches from a negative to positive value. (B) PLGA particle sizes increase as the zeta potential approaches a value near zero, indicating aggregation due to loss of electrostatic stability, then the PLGA particle sizes return to a normal range as the zeta potential increases above zero.

FIG. 6A-B: Adhesion of DPs to T24 bladder cancer cells. (A) confocal microscopy images of the adhesive properties of spherical microparticles and (B) PLGA DPs to exemplary T4 bladder cancer cells. Adhesive PLGA DPs with nanofibers results in stronger adhesion to cells than spherical microparticles, as evidenced by the detachment of cells in contact with the DPs from the plastic substrate.

FIG. 7: shows an exemplary embodiment where chitosan-functionalized PLGA DPs with chemotherapeutic drug can be extruded on tumor sites as they are incorporated in hydrogel. In one embodiment, an alginate gel alginate can form a DP-infused hydrogel. Alginate is a naturally occurring polymer obtained from seaweed and has been extensively investigated and used for biomedical applications, due to its biocompatibility and low toxicity. Application to cancerous bladder cells can be effective, since the alginate gel slowly dissolves in urine, DPs remain attached on epithelia allowing a sustainably release and targeted application of chemotherapeutic drugs.

FIG. 8A-C: Adhesion of PLGA DPs on bladder cells. (A) shows a scanning electron microscopy (SEM) image of DPs bound to a mouse bladder epithelium at 200 μm and 100 μm magnification, with additional identification of the sDPs. In this embodiment The cut bladder was treated with PLGA DPs and further washed by to simulate physiological shear stresses on epithelial cells in the bladder. Branched nanofibers around the PLGA microparticle increase van der Waals forces and enhances the adhesion of particles along soft tissue boundaries. (B) shows that as DP concentration in alginate gel increases, adhesives of the particles between glass slides increases; (C) demonstrates that the branched nanofibers of the DPs enhance the adhesion of the alginate gel between glass slides.

FIG. 9A-B: Chemotherapeutic effect of sDPs with encapsulated drugs. Shows that the chemical properties and solubility of a DP loaded with an exemplary chemotherapeutic agent can be altered by changing the solvent composition (e.g. ratio of THF and DMSO) resulting in the increased loading (B) and release (A) of the therapeutic agent.

FIG. 10A-B: T24 and HTB-9 cell viability for free drugs. Shows cell viability of T24 (A) and HTB-9 (B) cells at various dosages of exemplary free chemotherapeutic agents (gemcitabine, docetaxel, and methotrexate).

FIG. 11A-B: Cell viability for chitosan-functionalized drug-loaded DPs. Shows cell viability of T24 (A) and HTB-9 (B) cells at various dosages of exemplary chemotherapeutic agents administered via functionalized drug-loaded DPs. Data demonstrates sustainably released chemotherapeutic drugs from chitosan-functionalized PLGA sDPs effectively kill human cancer cells while the particles are adhered on the cells.

FIG. 12: schematic of drug-loaded DPs adhered to a target cell/tissue causing the sustained and targeted release of said drug.

FIG. 13: Effect of drug-loaded sDPs on HTB-9 cells. Alginate gel- and chitosan-functionalized DPs without drug show minimum toxicity on cancer cells, sustainably released gemcitabine from sDPs effectively kill cancer cells.

### DETAILED DESCRIPTION OF THE INVENTION

In one aspect, the invention includes a novel class of highly branched, nanostructured particles, generally referred to herein as dendritic particles (DPs), synthesized from biodegradable materials that can be specifically adapted for drug delivery. Due to their extremely high surface areas, the DPs of the invention robustly adhere to biological tissues for sustained periods, whereupon release therapeutic agents, such as therapeutic or prophylactic drugs, in a controlled and sustained fashion. As described below, the morphology of the DPs enables robust adhesion to biological surfaces (e.g., epithelial tissues, mucosa, skin, lining of organs) without the use of adhesives. The DPs can slowly release drugs for an extended period of time without becoming dislodged while breaking down into safe, biocompatible byproducts in physiological conditions.

In another aspect, the invention includes systems and methods for manufacturing DPs from biodegradable components, such that they can degrade over time into safe and biocompatible byproducts. In one preferred aspect, DPs are synthesized from poly(lactic-co-glycolic acid) (PLGA), which undergoes controlled degradation into biocompatible products (i.e., lactic acid, glycolic acid) under physiologically relevant conditions. Notably, PLGA is copolymer that is an FDA-approved material and widely used in therapeutic drug delivery due to its well-studied biocompatibility and biodegradability, as would be understood by one of ordinary skill in the art.

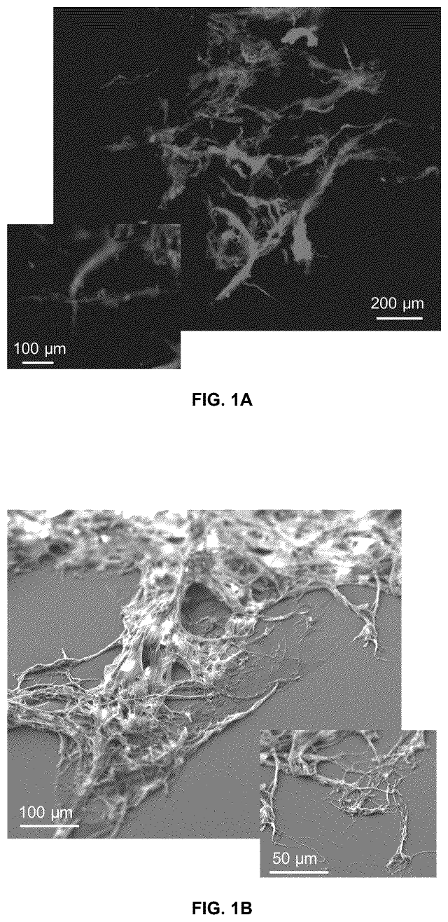

In one embodiment, the invention includes methods of synthesizing DPs, and preferably DPs having a quantity of embedded with a therapeutic or prophylactic agent. As detailed below, in this embodiment, in a highly turbulent flow, precipitation of the PLGA in an antisolvent results in the formation of branched nanofibers around the particle, which increases van der Waals forces and enhances the adhesion of particles along biological and other surfaces, and in particular soft tissue boundaries. The adhesive PLGA DPs result in stronger surface adhesion than spherical PLGA microparticles. As shown in FIGS. 1-3, due to strong van der Waals forces, the dendritic particles display retention on epithelial tissues such as a mouse bladder.

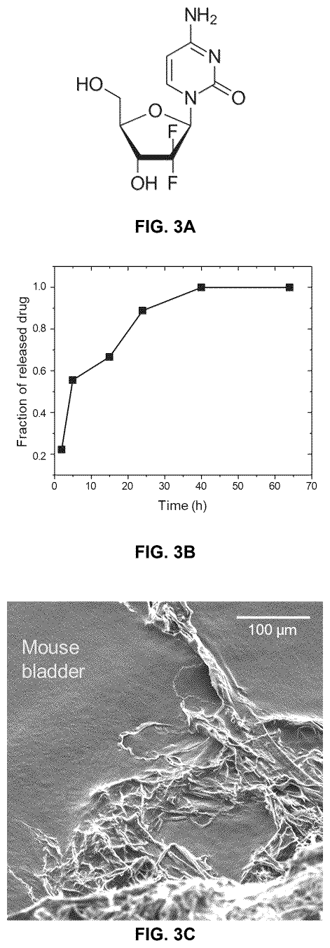

In one example, the present inventions synthesized a quantity of DPs embedded with an exemplary chemotherapeutic agent, in this case gemcitabine, which is an FDA-approved drug that treats various carcinomas. Specifically, gemcitabine is used as a first-line treatment alone for bladder cancer, pancreatic cancer, and metastatic non-small cell lung cancer. As shown in FIG. 3, due to their highly dendritic morphology, DPs strongly adhere to mouse bladder or epithelial tissues and gradually release the therapeutic compound over time. This sustained treatment can effectively kill cancer cells or activate immune cells sustainably over a longer period, while using fewer particles to achieve a therapeutically effective amount.

As notes above, under physiological conditions, the DPs are made from PLGA, such that the particles undergo a controlled degradation into biocompatible products (i.e., lactic acid, glycolic acid). In this manner, the DPs are able to remain in contact with the target cells/tissues and, as shown in FIG. 3, showed that sustained release of gemcitabine thereby treating, in this example, bladder cancer cells. These data demonstrate that DPs can act as a novel drug delivery strategy that offers high retention at target tissues for sustained drug release. Notably, the use of gemcitabine is exemplary only, and in no way should be seen as limiting. Indeed, the nature of the DPs of the invention are such that any therapeutic compounds or drug that can be dissolved in a solvent can be embedded in the DPs of the invention as described below.

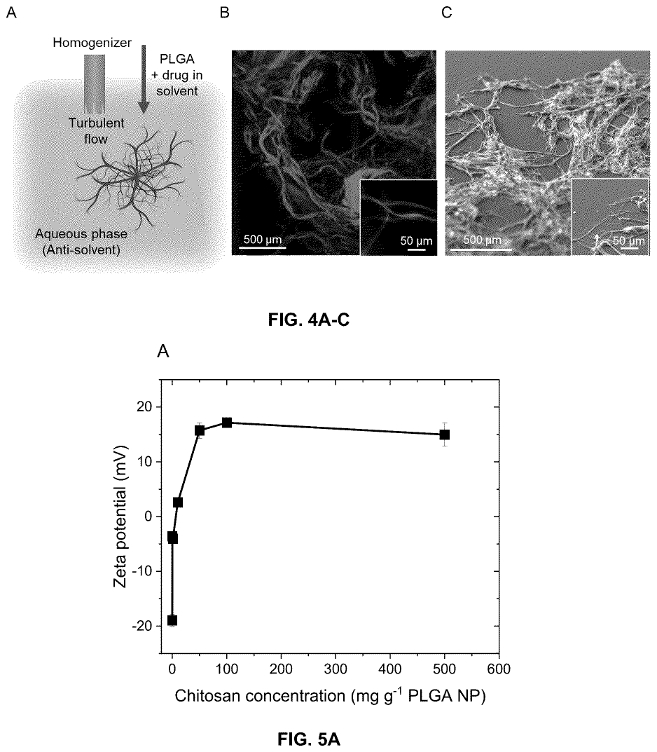

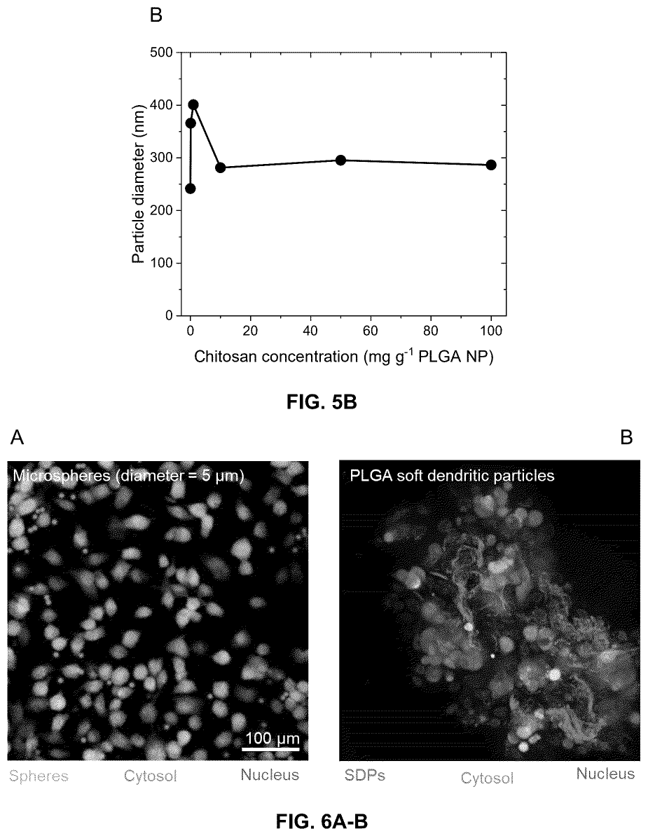

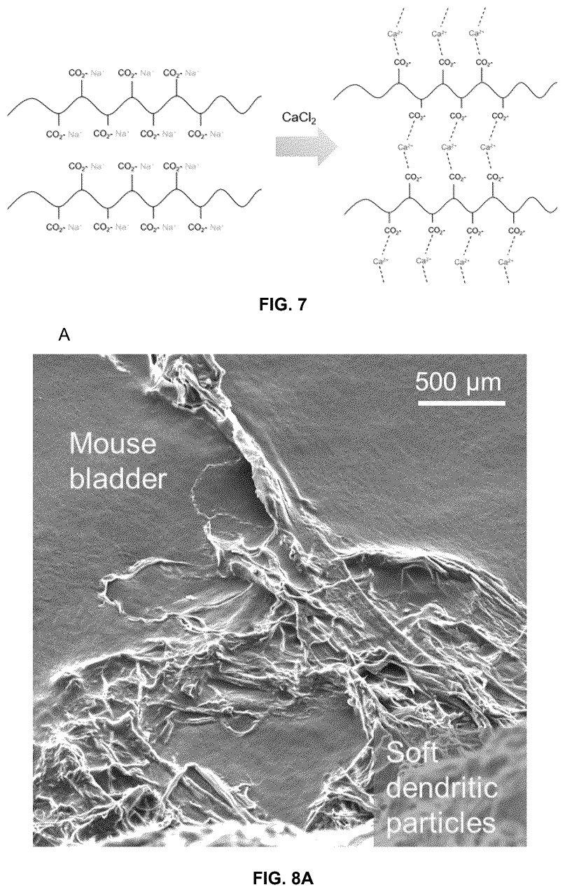

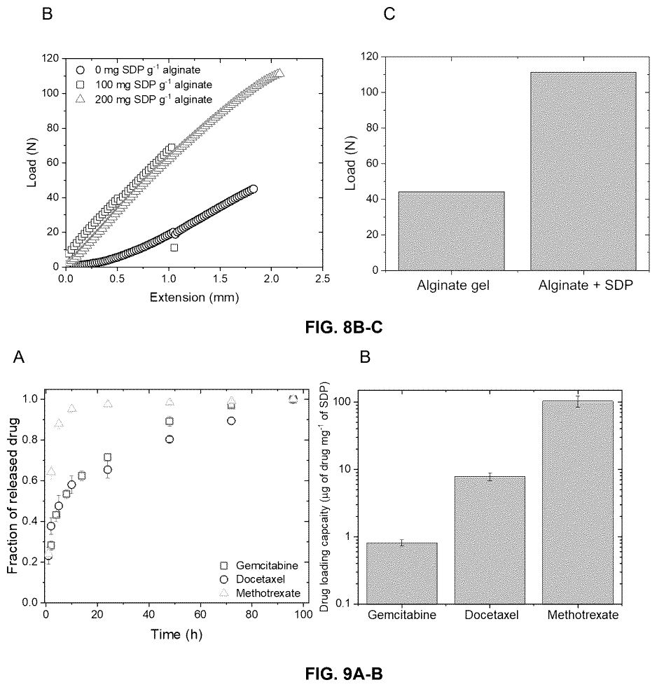

Methods of producing DPs of the invention, and preferably DPs embedded with one or more therapeutic compounds are included within the scope of the invention. In one embodiment shown in FIG. 4A, a DP embedded with more therapeutic compounds can be accomplished via fluid flow templating. Notably, production of the formation of DP, and in particular the formation of their dendritic structure includes factors such a turbulent flow of an aqueous phase (anti-solvent) loaded with a polymer and drug, the molecular weight of the polymer, and delayed precipitation for example as described by Velev et al., Nature Materials, 2019, 18, 1315-1320 | Advanced Materials, 2023, 35 (16), 2211438 (the process described therein for the formation of DPs are incorporated herein by reference).

A one preferred method is provided below, and DP can be formed by the following method:

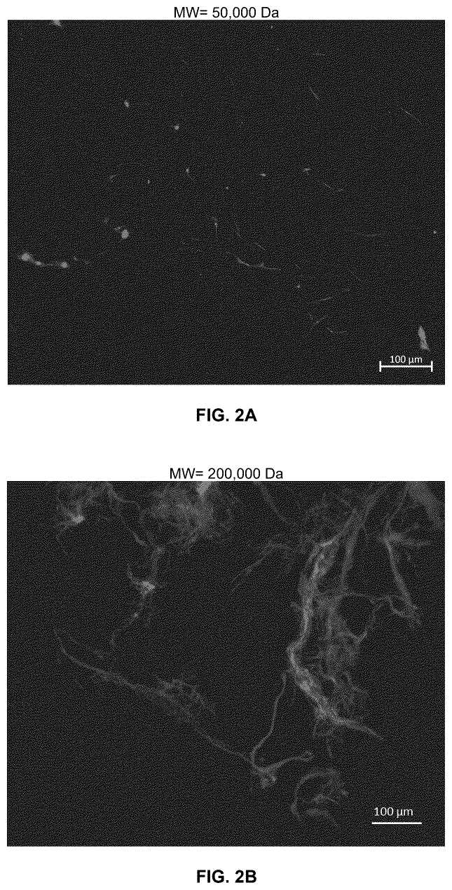

First, a solution of PLGA and a solvent is prepared. As shown in FIG. 2, in a preferred embodiment, a high molecular weight PLGA, such as like Resomer RG 858S (molecular weight 190,000-240,000 Da) can be mixed with a solvent. Notably, PLGA compounds with a significantly lower molecular weight are not capable of efficiently forming DPs of the invention. In a preferred example, a solution of 5 wt. % PLGA (Resomer RG 858S) in tetrahydrofuran (THF) is prepared.

In the present invention, “PLGA (poly (lactic-co-glycolic acid))” means a biodegradable polymer having the following structural formula:

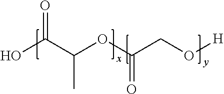

(Where x is the number of units of lactic acid and y is the number of units of glycolic acid). The PLGA is converted into lactic acid (lactic acid) and glycolic acid by hydrolysis and finally discharged into carbon dioxide and water. PLGA has been approved by the FDA as a drug carrier material with biodegradability and biocompatibility (e.g., bioabsorbable fracture plates, screw restorative membranes for dental materials, absorbent fins for pediatric tibia and fracture treatment, suture material), and it is widely used as a material for tissue engineering. The PLGA of the present invention is not limited by way of preferred embodiment, but may be commercially available, for example, from Sigma Aldrich. Notably, reference to PLGA is exemplary only, as other natural and synthetic biodegradable polymers can be suitable for the production of DPs of the invention. For example, natural biodegradable polymers may be used to synthesize DPs, which can include, but not be limited to: collagen, albumin, gelatin, and polysaccharides, such as agarose, alginate, carrageenan, hyaluronic acid (HA), dextran, chitosan, and cyclodextrins. Further, synthetic biodegradable polymers may be used to synthesize DPs, which can include, but not be limited to: α-hydroxy acids, polyanhydrides, poly (amides), poly (ester amides), poly (phosphoesters), poly (alkyl cyanoacrylates), poly (hyarulonic acids), and the like. Other suitable polymers would be known by those of ordinary skill in the art.

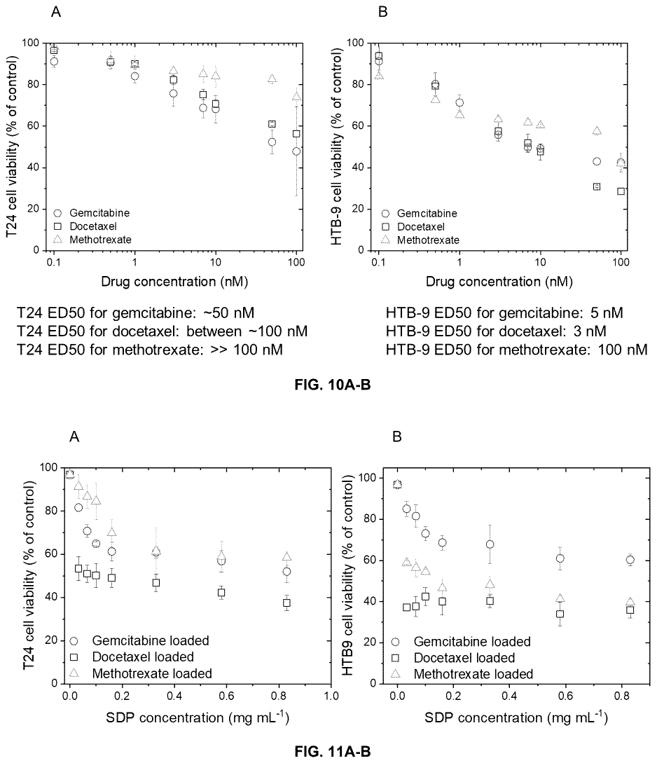

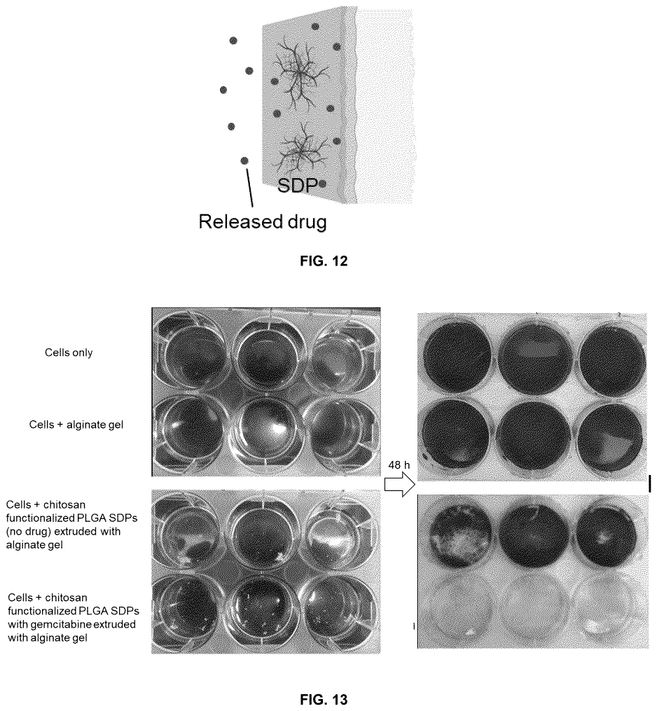

Second, a solution of one or more therapeutic compounds is also dissolved in a solvent. In the example shown in FIG. 3, a quantity of the chemotherapeutic agent gemcitabine is dissolved in a quantity of dimethylsurfoxide (DMSO). Additional chemotherapeutic agents, such as docetaxel and methotrexate loaded to a DP are further described in FIGS. 9-11 and 13.

Third, the solution of THF and DMSO solvents and their corresponding solutes are mixed, preferably with a 3:1 ratio of THF to DMSO.

Fourth, a quantity of THF/DMSO mixture is subjected to a sheer force in the presence of a nonionic detergent, such as a nonionic poloxamer. In a preferred embodiment, 1 mL of THF/DMSO mixture is injected into the shear zone of 25 mL of 1 wt. % Pluronic F108 aqueous solution in-one-go with the homogenizer setting at 30,000 RPM. Finally, the DPs containing a quantity of embedded gemcitabine is isolated.

In another embodiment, drug-loaded DPs of the invention can be loaded to a hydrogel and applied directly to a target biological site, such as a tumor, cancerous growth, tissue or organ. For example, in one embodiment a DP can be formed from PLGA as generally described herein and further functionalized by chitosan. The functionalized DPs can be loaded to a hydrogel, such as an alginate hydrogel which can be prepared, for example by extrusion, or other 3D printable application, for example as described by Williams, A. H., et al. Printable homocomposite hydrogels with synergistically reinforced molecular-colloidal networks. Nat Commun 12, 2834 (2021)(the printable hydrogel methods described therein being incorporate in their entirety by refence). As used herein, the term “hydrogel,” means a hydrophilic or amphiphilic polymeric network composed of homopolymers or copolymers, which is insoluble due to the presence of covalent chemical crosslinks. The crosslinks provide the network structure and physical integrity. Hydrogels exhibit a thermodynamic compatibility with water which allows them to swell in aqueous media. In a preferred aspect, a hydrogel can be a pharmaceutically acceptable carrier for the DPs of the invention.

Additional aspect of the invention include methods and compositions for treating a subject in need thereof. In this preferred embodiment, a therapeutically effective amount of DPs embedded with a therapeutic agent are administered to a subject in need thereof. In this preferred embodiment, the subject includes an animal, and preferably a human having a disease or condition, such as cancer. Again, in this preferred embodiment, DPs embedded with a therapeutic agent can include DPs embedded with a chemotherapeutic agent, such as gemcitabine, a therapeutically effective amount of which can be administered to a subject in need thereof.

Additional aspects of the invention include pharmaceutical compositions for treating a subject in need thereof. In this preferred aspect, a pharmaceutical composition of the invention includes a therapeutically effective amount of DPs embedded with a therapeutic agent and a pharmaceutically acceptable carrier, which are administered to a subject in need thereof. In one embodiment, the pharmaceutical composition of the invention includes a therapeutically effective amount of DPs embedded with a therapeutic agent loaded to a hydrogel that can be applied directly, or indirectly to a target area, such as a biological site of interest, tumor, cancerous cells, tissue or organ.

Additional aspects of the invention include a kit, including pharmaceutical compositions for treating a subject in need thereof. In this preferred aspect, a kit of the invention includes a pharmaceutical composition comprising a therapeutically effective amount of DPs embedded with a therapeutic agent and a pharmaceutically acceptable carrier, a container for holding the composition and instructions for use.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

As used herein, a “a dendritic particle,” or “dendritic nanoparticle” means a particle numerous nanostructured branches.

The term “isolated,” when applied to a particle, and in particular DP of the invention, denotes that the particle is essentially free of other components with which it is associated in a natural state, or prior to, or during its synthesis. It is preferably in a homogeneous state and can be in either a dry or aqueous solution. The term “isolated” denotes that particle is at least 85% pure, more preferably at least 95% pure, and most preferably at least 99% pure, or greater than 99% pure.

The terms “embedding” in the context of the present invention with respect to embedding one or more compounds with a DP to form a complex refers to joining, associating, encapsulating, or otherwise conjugating one or more compounds within the matrix of a DP.

The term “compound,” “agent,” or “therapeutic compound or agent,” includes any chemical agent, preferably a small-molecule drug or prodrug, and even more preferably a chemotherapeutic agent which includes all solvates, complexes, polymorphs, radiolabeled derivatives, tautomers, stereoisomers, and optical isomers of the same unless otherwise specified.

A “cancer therapy” may include administration of a therapeutically effective amount of a “chemotherapeutic agent,” which is meant one or more chemical agents used in the treatment or control of proliferative diseases (e.g., cancer). Chemotherapeutic agents include cytotoxic and cytostatic agents. Exemplary chemotherapeutic agents may mediate DNA damage (e.g., alkylating chemotherapeutic agents). Non-limiting examples of chemotherapeutic agents are generally known in the art. In a preferred embodiment, the chemotherapeutic agent is embedded in, and delivered to a cell or tissue in a DP of the invention.

As used herein, “pharmaceutical compositions” are compositions that include an amount (for example, a unit dosage) of the disclosed DPs, which preferably include an embedded therapeutic compound(s), together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

Cosolvents and adjuvants may be added to the pharmaceutical compositions of the invention. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone. Supplementary active compounds (e.g., preservatives, antioxidants, antimicrobial agents including biocides and biostats such as antibacterial, antiviral, and antifungal agents) can also be incorporated into the compositions. Preservatives and other additives include, for example, antimicrobials, anti-oxidants, chelating agents, and inert gases (e.g., nitrogen). Pharmaceutical compositions may therefore include preservatives, antimicrobial agents, anti-oxidants, chelating agents, and inert gases. Preservatives can be used to inhibit microbial growth or increase stability of the active ingredient thereby prolonging the shelf life of the pharmaceutical compositions. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles, or filling agents) suitable for administration to any cell, tissue, or organ, in vivo, ex vivo (e.g., tissue or organ transplant) or in vitro, by various routes and delivery, locally, regionally, or systemically.

Exemplary routes of administration for contact or in vivo delivery include a dosage of DPs that is sufficient to deliver to a target cell or tissue a therapeutic compound that achieves a desired therapeutic effect. Routes of administration of the DPs of the invention can be formulated include inhalation, respiration, intubation, bladder instillation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

Further aspects of the pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

As used herein, “therapeutically effective amount” of the disclosed invention includes a quantity of DPs having an embedded therapeutic compound(s) that provides a dosage of the compound that is sufficient to achieve a desired therapeutic effect, such as treatment of a disease or disorder such as cancer. In some examples, a therapeutically effective amount is an amount sufficient to achieve tissue concentrations at the site of action that are similar to those that are shown to have a therapeutic effect in the subject, or cell/tissue. For example, a therapeutically effective amount of a DPs having an embedded therapeutic compound(s), may be such that the subject receives a dosage of less than or about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day, or more.

The terms “individual,” “subject,” and “patient,” used interchangeably herein, refer to a mammal, including, but not limited to, murines, simians, humans, mammalian farm animals, mammalian sport animals, and mammalian pets. Preferably, the subject herein is human. As used herein, the phrase “in need thereof” means that the animal or mammal has been identified as having a need for the particular method or treatment. In some embodiments, the identification can be by any means of diagnosis. In any of the methods and treatments described herein, the animal or mammal may be in need thereof. In some embodiments, the animal or mammal is in an environment or will be traveling to an environment in which a particular disease, disorder, or condition is prevalent.

As used herein, the term “cancer” is meant a disease characterized by the pathological proliferation of a cell or tissue and its subsequent migration to or invasion of other tissues or organs. Cancer growth is typically uncontrolled and progressive, and occurs under conditions that would not elicit, or would cause cessation of, multiplication of normal cells. Cancers can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Non-limiting examples of cancers include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

By “treating” a disease, disorder, or condition further means delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., cancer) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives.

In one embodiment, treating cancer means reducing the “symptoms of cancer,” by which is meant one or more (e.g., one, two, three, four, or five) of the physical manifestations of cancer. Non-limiting examples of symptoms of cancer include blood in urine, pain or burning upon urination, cloudy urine, pain in bone, fractures in bones, fatigue, weight loss, repeated infections, nausea, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

In the methods of the invention, dose of DPs, which preferably include an embedded therapeutic compound(s), may be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly, or monthly. Timing of contact, administration ex vivo or in vivo delivery can be dictated by the pathogenesis, symptom, pathology, or adverse side effect to be treated. For example, an amount can be administered to the subject substantially contemporaneously with, or within about 1-60 minutes or hours of the onset of a symptom or adverse side effect of treatment.

Doses may vary depending upon whether the treatment is therapeutic or prophylactic, the onset, progression, severity, frequency, duration, probability of or susceptibility of the symptom, the type of pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Doses can be based upon current existing treatment protocols, empirically determined, determined using animal disease models or optionally in human clinical studies. A subject may be administered in single bolus or in divided/metered doses, which can be adjusted to be more or less according to the various consideration set forth herein and known in the art. Dose amount, frequency or duration may be increased or reduced, as indicated by the status of pathogenesis, associated symptom or pathology, or any adverse side effect(s). For example, once control or a particular endpoint is achieved, for example, reducing, decreasing, inhibiting, ameliorating, or preventing onset, severity, duration, progression, frequency, or probability of one or more symptoms associated with a telomere-associated disease or disorder. Another embodiment of this disclosure provides pharmaceutical kits containing a pharmaceutical composition of this disclosure containing a DPs, which preferably include an embedded therapeutic compound(s), prescribing information and/or directions for the use of the composition, and a container.

In another preferred aspect, the present invention provides a method for treating a disease or condition, and preferably cancer, comprising: administering to a subject in need thereof, a therapeutically effective amount of DPs including an embedded therapeutic compound(s), or a pharmaceutical composition containing a therapeutically effective amount of DPs including an embedded therapeutic compound(s) and a pharmaceutically acceptable carrier.

In one embodiment, the therapeutic compound(s) of the invention include chemotherapeutic agent, which may preferably be embedded in the DPs of the invention. By “chemotherapeutic agent” is meant one or more chemical agents used in the treatment or control of proliferative diseases (e.g., cancer). Chemotherapeutic agents include cytotoxic and cytostatic agents. Exemplary chemotherapeutic agents may mediate DNA damage (e.g., alkylating chemotherapeutic agents). Non-limiting examples of chemotherapeutic agents are described herein and are known in the art. Desirably, the chemotherapeutic agent administered induces apoptosis or necrosis of the cancer cells. Non-limiting examples of chemotherapeutic agents useful in these methods include: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

For cancer treatment, depending on the type of cancer and its stage of development, the therapy can be used to slow the spread of the cancer, to slow the cancer's growth, to kill or arrest cancer cells that may have spread to other parts of the body from the original tumor, or to relieve symptoms caused by the cancer. A skilled physician may monitor the effectiveness of treatment of a cancer by monitoring the severity or duration of one or more symptoms of cancer. Non-limiting examples of symptoms of cancer include: blood in urine, pain or burning upon urination, cloudy urine, pain in bone, fractures in bones, fatigue, weight loss, repeated infections, nausea, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

1. A drug-delivery system comprising a dendritic particle (DP) having a plurality of nanostructured branches embedded with one or more therapeutic compounds configured to adhere to a target tissue and release the compounds to the target, wherein the particle is prepared from a polymer that is biodegradable at physiological conditions.

2. The system of claim 1, wherein said polymer is selected from: poly(lactic-co-glycolic acid) (PLGA), collagen, albumin, gelatin, and polysaccharides, such as agarose, alginate, carrageenan, hyaluronic acid (HA), dextran, chitosan, cyclodextrins, α-hydroxy acids, polyanhydrides, poly (amides), poly (ester amides), poly (phosphoesters), poly (alkyl cyanoacrylates), and poly (hyarulonic acids).

3. The system of claim 2, wherein said PLGA comprise a high molecular weight PLGA.

4. The system of claim 3, wherein said high molecular weight PLGA comprises PLGA having a molecular weight of at least 20 kDa, or more.

5. The system of claim 1, wherein said physiological conditions comprises physiological conditions present in a subject in vivo.

6. The system of claim 5, wherein said target tissue comprises the epithelia of the subject, or mucosa of a subject, or lining of one or more organs of a subject, or a cell of an organ or tissue of a subject, or a combination of the aforementioned.

7. The system of claim 6, wherein said epithelia of the subject comprises bladder epithelia of the subject.

8. The system of claim 1, wherein said one or more therapeutic compounds comprise one or more chemotherapeutic agents.

9. The system of claim 8, wherein said one or more one or more chemotherapeutic agents is selected from: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine, or a combination of the aforementioned.

10. A pharmaceutical composition comprising the DP of any of claims 1-9, and a pharmaceutically acceptable carrier.

11. The pharmaceutical composition of claim 10, wherein said pharmaceutically acceptable carrier comprises a hydrogel.

12-32. (canceled)

33. A kit comprising the pharmaceutical composition of claim 10, a container, and instructions for use.

34. (canceled)
